(19) United States
(12) Patent Application Publication
(10) Pub. No.: US 2026/0183542 A1
(43) Pub. Date: Jul. 2, 2026

(54) Automatic Pairing of Electrode Assemblies That Are Used to Apply Tumor Treating Fields (TTFields) to a Subject's Body Based on Responses to Signals That Are Sent Into the Electrode Assemblies

(71) Applicant: Novocure GmbH, Baar (CH)

(72) Inventor: Kathryn SMITH-PETERSEN, Carlisle, MA (US)

(73) Assignee: Novocure GmbH, Baar (CH)

(21) Appl. No.: 19/436,024

(22) Filed: Dec. 30, 2025

Related U.S. Application Data
(60) Provisional application No. 63/740,921, filed on Dec. 31, 2024.

(51) Int. Cl. A61N 1/36; A61N 1/02; A61N 1/04

(52) U.S. Cl. CPC A61N 1/36002; A61N 1/025; A61N 1/0408; A61N 1/0496; A61N 1/36025; A61N 1/3603

(57) ABSTRACT

Alternating electric fields (e.g., TTFields) can be imposed in a subject's body using a set of electrode assemblies. Each of the electrode assemblies is connected to a switch array by a respective cable. The system sends signals into these cables (e.g., signals that cause LEDs mounted on the electrode assemblies to light up) and observes the effects of sending the signals (e.g., observing which LED lights up). Based on the observation, the system determines which of the electrode assemblies are positioned on opposite sides of a region of interest in the subject's body. The system can then configure the switch array to apply an AC voltage across those electrode assemblies to induce TTFields in the subject body.

### CROSS REFERENCE TO RELATED APPLICATIONS

This Application claims the benefit of U.S. Provisional Application 63/740,921, filed Dec. 31, 2024, which is incorporated herein by reference in its entirety.

### BACKGROUND

Tumor Treating Fields (TTFields) therapy is a proven approach for treating tumors using alternating electric fields at frequencies e.g., between 50 kHz-1 MHz, more commonly 100-500 KHz. FIG. 1 depicts the prior art Optune® system, which delivers TTFields to patients via four electrode assemblies 90 (a.k.a. transducer arrays) that are placed on the patient's skin near the tumor. These electrode assemblies are arranged in two pairs, e.g., with one pair of electrode assemblies positioned to the left and right of the tumor, and the other pair of electrode assemblies positioned anterior and posterior to the tumor. Each electrode assembly is connected via a multi-wire cable to an AC signal generator 95. The AC signal generator 95 (a) sends an AC current through the anterior/posterior (A/P) pair of electrode assemblies 90 for 1 second, which induces an electric field with a first direction through the tumor; then (b) sends an AC current through the left/right (L/R) pair of electrode assemblies 90 for 1 second, which induces an electric field with a second direction through the tumor; then repeats steps (a) and (b) for the duration of the treatment.

Optune® will not work properly unless each pair of electrode assemblies are placed on the appropriate location(s) on a patient's body (e.g., with the L/R electrode assemblies positioned on opposite sides of the tumor, and with the A/P electrode assemblies positioned on opposite sides of the tumor). To help ensure that this occurs, the connectors on the electrode assemblies 90 are color-coded (i.e., black for the A/P electrode assemblies, and white for the L/R electrode assemblies) so that they will only mate with the corresponding connector on the AC signal generator 95.

### SUMMARY OF THE INVENTION

One aspect of the invention is directed to a first method of configuring a system that includes at least four electrode assemblies positioned on or in a subject's body. Each of the electrode assemblies is connected via a respective cable to a switch array. The first method comprises sending at least one first signal into a first subset of the cables; observing at least one first effect of the sending of the at least one first signal; sending at least one second signal into a second subset of the cables; observing at least one second effect of the sending of the at least one second signal; sending at least one third signal into a third subset of the cables; observing at least one third effect of the sending of the at least one third signal; and determining, based on the observed first, second, and third effects, which of the electrode assemblies are positioned on opposite sides of a region of interest in the subject's body.

Some instances of the first method further comprise configuring the switch array to route an AC voltage between whichever electrode assemblies were determined to be positioned on opposite sides of the region of interest. Optionally, these instances can further comprise applying the AC voltage between whichever electrode assemblies were determined to be positioned on opposite sides of the region of interest.

Some instances of the first method further comprise (a) applying an AC voltage between a first pair of the electrode assemblies that was determined to be positioned on opposite sides of the region of interest; (b) applying an AC voltage between a second pair of the electrode assemblies that was determined to be positioned on opposite sides of the region of interest; and (c) repeating step (a) and step (b) in an alternating sequence at least 100 times.

Some instances of the first method further comprise sending at least one fourth signal into a fourth subset of the cables; and observing at least one fourth effect of the sending of the at least one fourth signal. The determining is based on the observed first, second, third, and fourth effects.

In some instances of the first method, the determining is based on detecting light that is emitted in response to the sending of the at least one first signal, the at least one second signal, and the at least one third signal.

In some instances of the first method, each of the electrode assemblies includes a respective light source, the sending of the at least one first signal into the first subset of the cables comprises activating a first single one of the light sources via a first single one of the cables, the observing of the at least one first effect comprises observing which of the light sources is emitting light, the sending of the at least one second signal into the second subset of the cables comprises activating a second single one of the light sources via a second single one of the cables, the observing of the at least one second effect comprises observing which of the light sources is emitting light, the sending of the at least one third signal into the third subset of the cables comprises activating a third single one of the light sources via a third single one of the cables, and the observing of the at least one third effect comprises observing which of the light sources is emitting light.

In some instances of the first method, each of the electrode assemblies includes a respective light source, the sending of the at least one first signal into the first subset of the cables comprises activating a first single one of the light sources via a first single one of the cables, the observing of the at least one first effect comprises observing which of the light sources is emitting light, the sending of the at least one second signal into the second subset of the cables comprises activating a second single one of the light sources via a second single one of the cables, the observing of the at least one second effect comprises observing which of the light sources is emitting light, the sending of the at least one third signal into the third subset of the cables comprises activating a third single one of the light sources via a third single one of the cables, the observing of the at least one third effect comprises observing which of the light sources is emitting light, the observing is implemented by capturing a plurality of images of at least some of the electrode assemblies using at least one camera, and the determining is implemented by analyzing the captured plurality of images.

Optionally, the instances described in the previous paragraph can further comprise sending at least one fourth signal into a fourth subset of the cables, and observing at least one fourth effect of the sending of the at least one fourth signal. In these instances, the determining is based on the observed first, second, third, and fourth effects. The sending of the at least one fourth signal into the fourth subset of the cables comprises activating a fourth single one of the light sources via a fourth single one of the cables. And the observing of the at least one fourth effect comprises observing which of the light sources is emitting light.

In some instances of the first method, the determining is based on measuring impedances or conductances that are encountered by the at least one first signal, the at least one second signal, and the at least one third signal.

In some instances of the first method, the sending of the at least one first signal into the first subset of the cables comprises applying a first electrical signal between a first single one of the cables and a second single one of the cables, and the observing of at least one first effect of the sending of the at least one first signal comprises measuring an impedance that is encountered by the first electrical signal or measuring a conductance that is encountered by the first electrical signal. The sending of the at least one second signal into the second subset of the cables comprises applying a second electrical signal between the first single one of the cables and a third single one of the cables, and the observing of at least one second effect of the sending of the at least one second signal comprises measuring an impedance that is encountered by the second electrical signal or measuring a conductance that is encountered by the second electrical signal. And the sending of the at least one third signal into the third subset of the cables comprises applying a third electrical signal between the first single one of the cables and a fourth single one of the cables, and the observing of at least one third effect of the sending of the at least one third signal comprises measuring an impedance that is encountered by the third electrical signal or measuring a conductance that is encountered by the third electrical signal.

In some instances of the first method, the determining comprises identifying a highest one of the measured impedances or a lowest one of the measured conductances, and determining that two of the cables that correspond to the identifying lead to electrode assemblies that are positioned on opposite sides of the region of interest. Optionally, in these instances, there can be exactly four electrode assemblies positioned on or in the subject's body and exactly four cables, and the determining further comprises determining that two of the cables that do not correspond to the identifying also lead to electrode assemblies that are positioned on opposite sides of the region of interest.

Another aspect of the invention is directed to a first apparatus for applying alternating electric fields to a region of interest within a subject's body. The first apparatus includes a controller, an AC signal generator, a switch array, and a circuit. The AC signal generator generates an output at a frequency between 50 kHz and 1 MHz. The switch array has (i) two inputs that accept the output of the AC signal generator and (ii) first, second, third, and fourth output connectors. The circuit is configured to send auxiliary signals into the first, second, third, and fourth output connectors when electrode assemblies are connected to the first, second, third, and fourth output connectors via first, second, third, and fourth cables, respectively. The switch array is dynamically configurable to, based on at least one control signal that arrives from the controller, route the output of the AC signal generator to either (a) the first and second output connectors, (b) the first and third output connectors, (c) the first and fourth output connectors, (d) the second and third output connectors, (e) the second and fourth output connectors, or (f) the third and fourth output connectors. And the controller is configured to (a) accept input data indicative of effects of the sending of the auxiliary signals into the first, second, third, and fourth output connectors, (b) determine, based on the input data, which of the electrode assemblies are positioned on opposite sides of a region of interest in the subject's body, and (c) control the switch array so that the switch array routes the output of the AC signal generator to connectors that lead, via corresponding cables, to electrode assemblies that are positioned on opposite sides of the region of interest.

In some embodiments of the first apparatus, the controller is configured to (i) control the switch array so that the switch array routes the output of the AC signal generator to connectors that lead, via corresponding cables, to a first pair of the electrode assemblies that are positioned on opposite sides of the region of interest; (ii) control the switch array so that the switch array routes the output of the AC signal generator to connectors that lead, via corresponding cables, to a second pair of the electrode assemblies that are positioned on opposite sides of the region of interest; and repeat steps (i) and (ii) in an alternating sequence.

In some embodiments of the first apparatus, the auxiliary signals that are sent into the first, second, third, and fourth output connectors are configured to activate respective light sources that are positioned on the electrode assemblies. Optionally, in these embodiments, the accepted input data represent at least one image of the electrode assemblies.

In some embodiments of the first apparatus, the auxiliary signals that are sent into the first, second, third, and fourth output connectors are configured to obtain impedance measurements or conductance measurements, and the accepted input data represent impedance measurements or conductance measurements.

Another aspect of the invention is directed to a first electrode assembly that includes a flex circuit, at least one intermediate layer, a front layer of biocompatible conductive adhesive or biocompatible conductive hydrogel, at least one temperature sensor, a light source, and a flexible cable. The flex circuit includes at least one conductive pad disposed on a front side of the flex circuit. The at least one intermediate layer is disposed in front of the flex circuit. The front layer of biocompatible conductive adhesive or biocompatible conductive hydrogel is disposed in front of the at least one intermediate layer, and the front layer is configured to adhere to a person's skin. The at least one temperature sensor is disposed in thermal contact with the at least one conductive pad. The light source is aimed to emit light rearwards with respect to the flex circuit. The flexible cable has (a) a multi-pin connector, (b) a first conductor configured to route first electrical signals from a first pin of the connector to the at least one conductive pad, wherein the first electrical signals are configured to, when at least two electrode assemblies have been affixed to a subject's body, induce an alternating electric field in a region of interest in the subject's body, (c) at least one second conductor configured to route second electrical signals from the at least one temperature sensor to at least one second pin of the connector, and (d) at least one third conductor configured to route third electrical signals from at least one third pin of the connector to the light source, wherein the third electrical signals are configured to cause the light source to emit light.

In some embodiments of the first electrode assembly, the at least one intermediate layer comprises: a sheet of graphite disposed in front of the flex circuit, wherein the sheet of graphite has a front face and a rear face; and a layer of conductive adhesive disposed between the at least one conductive pad and the rear face of the sheet of graphite, in electrical contact with both the at least one conductive pad and the rear face of the sheet of graphite.

In some embodiments of the first electrode assembly, the at least one intermediate layer comprises a layer of flexible polymer material having a dielectric constant of at least 10.

Another aspect of the invention is directed to a second electrode assembly that comprises a flex circuit, a sheet of graphite, a layer of conductive adhesive, a front layer of biocompatible conductive adhesive or biocompatible conductive hydrogel, at least one temperature sensor, a light source, and a flexible cable. The flex circuit includes at least one conductive pad disposed on a front side of the flex circuit. The sheet of graphite is disposed in front of the flex circuit, and the sheet of graphite has a front face and a rear face. The layer of conductive adhesive is disposed between the at least one conductive pad and the rear face of the sheet of graphite, in electrical contact with both the at least one conductive pad and the rear face of the sheet of graphite. The front layer of biocompatible conductive adhesive or biocompatible conductive hydrogel is disposed in front of the sheet of graphite, and the front layer is configured to adhere to a person's skin. The at least one temperature sensor is disposed in thermal contact with the sheet of graphite. The light source is aimed to emit light rearwards with respect to the flex circuit. The flexible cable has (a) a multi-pin connector, (b) a first conductor configured to route first electrical signals from a first pin of the connector to the at least one conductive pad, wherein the first electrical signals are configured to, when at least two electrode assemblies have been affixed to a subject's body, induce an alternating electric field in a region of interest in the subject's body, (c) at least one second conductor configured to route second electrical signals from the at least one temperature sensor to at least one second pin of the connector, and (d) at least one third conductor configured to route third electrical signals from at least one third pin of the connector to the light source, wherein the third electrical signals are configured to cause the light source to emit light.

Another aspect of the invention is directed to a third electrode assembly that comprises at least one electrode element, a layer of conductive adhesive or conductive hydrogel, at least one temperature sensor, a light source, and a flexible cable. The at least one electrode element is positioned and oriented to apply alternating electric fields to a subject's body. The layer of conductive adhesive or conductive hydrogel is (i) positioned in front of the at least one electrode element and (ii) configured to adhere to a subject's body. The at least one temperature sensor is disposed in direct and/or indirect thermal contact with the at least one electrode element. The light source is positioned and oriented so that light emitted by the light source can be seen by observers in a vicinity of the subject while the electrode assembly is positioned on the subject's body. The flexible cable has (a) a multi-pin connector, (b) a first conductor configured to route first electrical signals from a first pin of the connector to the at least one electrode element, wherein the first electrical signals are configured to, when at least two electrode assemblies have been affixed to a subject's body, induce an alternating electric field in a region of interest in the subject's body, (c) at least one second conductor configured to route second electrical signals from the at least one temperature sensor to at least one second pin of the connector, and (d) at least one third conductor configured to route third electrical signals from at least one third pin of the connector to the light source, wherein the third electrical signals are configured to cause the light source to emit light.

Some embodiments of the third electrode assembly further comprise at least one intermediate layer of material positioned between the at least one electrode element and the layer of conductive adhesive or conductive hydrogel.

Optionally, in the embodiments described in the previous paragraph, the at least one intermediate layer can comprise (a) a sheet of graphite disposed in front of the at least one electrode element, wherein the sheet of graphite has a front face and a rear face; and (b) a layer of conductive adhesive disposed between the at least one electrode element and the rear face of the sheet of graphite, in electrical contact with both the at least one electrode element and the rear face of the sheet of graphite. Optionally, in the embodiments described in the previous paragraph, the at least one intermediate layer can comprise a layer of flexible polymer material having a dielectric constant of at least 10.

In some embodiments of the third electrode assembly, the at least one electrode element comprises a conductive pad that is disposed on a front side of a flex circuit.

### BRIEF DESCRIPTION OF THE DRAWINGS

FIG. 1. In a simplified block diagram of a prior art system for delivering TTFields to a subject's body.

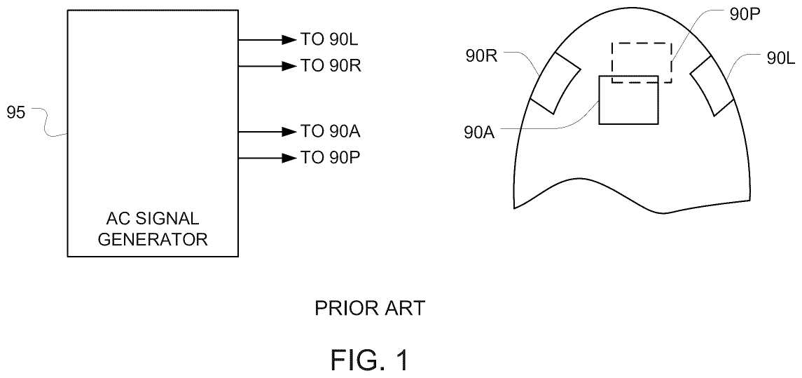

FIG. 2 is a block diagram of a first embodiment that dynamically configures how alternating voltages are applied to a set of electrode assemblies based on impedance measurements.

FIG. 3 depicts the FIG. 2 embodiment with a different arrangement of the cables that run between the switch array and the electrode assemblies.

FIG. 4 is a block diagram of a second embodiment that dynamically configures how alternating voltages are applied to a set of electrode assemblies based on signals that cause LEDs mounted on the electrode assemblies to illuminate.

FIG. 5 depicts the FIG. 4 embodiment with a different arrangement of the cables that run between the switch array and the electrode assemblies.

FIGS. 6 and 7 depict plan and section views of one example of how to implement the electrode assemblies of the FIG. 4 embodiment.

Various embodiments are described in detail below with reference to the accompanying drawings, wherein like reference numerals represent like elements.

### DESCRIPTION OF THE PREFERRED EMBODIMENTS

Although the prior art approach of color coding the connectors on the electrode assemblies is workable, problems can arise if a subject ignores the color coding and positions the prior art electrode assemblies improperly. Assume, for example, that a subject (1) positions the two electrode assemblies with black connectors on the anterior and left sides of their head, (2) positions the two electrode assemblies with white connectors on the posterior and right sides of their head, and (3) subsequently plugs the white and black connectors into the correspondingly-colored ports of the AC signal generator. This scenario will cause the system to apply AC voltages (a) between the anterior and left electrode assemblies, and (b) between the posterior and right electrode assemblies. The TTFields that are generated using this setup will typically be relatively weak in the middle section of the subject's head, with an associated reduction in the efficacy of treatment. In addition, because the AC voltages are being applied between neighboring electrode assemblies (rather than opposite electrode assemblies), the adjacent edges of the electrode assemblies will begin to heat up, which will cause the prior art system to reduce its output current to prevent overheating. And this reduction in current will further limit the efficacy of treatment. Furthermore, because all four of the electrode assemblies are not identical, they are non-interchangeable, and must be provided to the subject in sets.

With the methods and embodiments described herein, the system can determine the relative locations of the electrode assemblies that have been positioned on the subject's body, and dynamically configure how alternating voltages are applied to those electrode assemblies based on the determined relative locations. These methods and embodiments advantageously ensure that the electrode assemblies will never be improperly positioned or improperly connected to the AC signal generator. They also advantageously allow all four electrode assemblies to be interchangeable, which means that the subject does not have to be as careful when they position the electrode assemblies on their body.

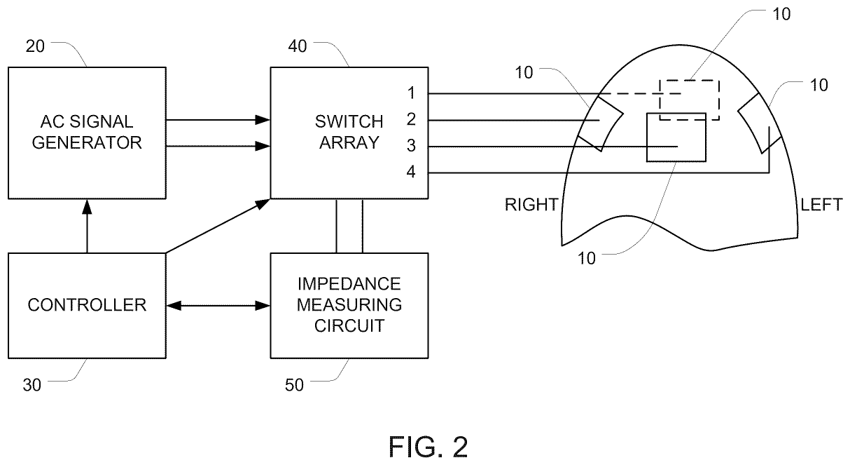

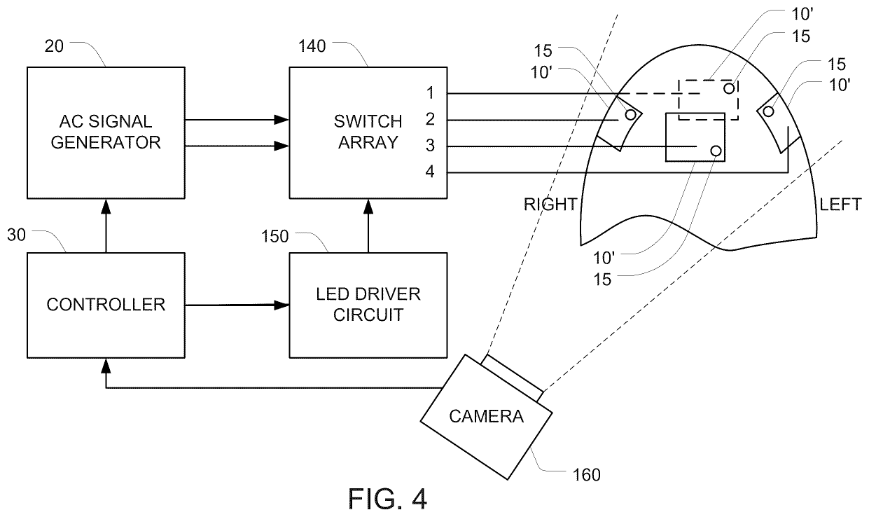

FIGS. 2 and 4 are block diagrams of two different embodiments that dynamically configure how alternating voltages are applied to a set of electrode assemblies that are positioned on a subject's body. Both of these embodiments do this by determining which electrode assemblies are positioned on opposite sides of a region of interest (ROI) in the subject's body, and subsequently configuring things so that an AC voltage is applied between electrode assemblies that are positioned on opposite sides of the ROI. In both the FIG. 2 and FIG. 4 embodiments, the system accomplishes this by sending signals into the cables that feed the electrode assemblies, observing the effects of sending those signals, and determining which electrode assemblies are positioned on opposite sides of the ROI based on the observed effects.

FIG. 2 is a block diagram of a first embodiment that relies on impedance measurements to dynamically configure how alternating voltages are applied to a set of electrode assemblies 10 that are positioned on a subject's body.

A wide variety of electrode assemblies can be used in the implementations described herein, including but not limited to the electrode assemblies described in U.S. Pat. No. 8,715,203, US 2023/0043071, and US 2021/0402179, each of which is incorporated herein by reference in its entirety. The electrode assemblies 10 can be held to the skin, e.g., using a conductive adhesive or a conductive hydrogel disposed on the front face of each electrode assembly. Optionally, the electrode assemblies 10 can also be secured to the subject's skin around their periphery e.g., using a flexible fabric with a self-adhesive front surface that resembles a Band-Aid® brand adhesive bandage.

An AC signal generator 20 generates an AC voltage at 50 kHz-1 MHz (e.g., 150-250 kHz, 100-300 kHz, 100-250 kHz, 150-300 kHz, or 75-125 kHz). This AC signal generator 20 can be built using a variety of approaches that will be apparent to persons skilled in the relevant arts, including but not limited to the AC signal generator described in U.S. Pat. No. 9,910,453 (modified to have a single output channel that remains active 100% the time).

A switch array 40 has two primary input terminals and four output connectors, and it routes the signals that arrive (from the AC generator 20) at the two primary input terminals to either (a) the first and second output connectors, (b) the first and third output connectors, (c) the first and fourth output connectors, (d) the second and third output connectors, (e) the second and fourth output connectors, or (f) the third and fourth output connectors. The switch array 40 implements one of the options (a)-(f) described in the previous sentence based on at least one control signal that arrives from a controller 30.

The switch array 40 also has two auxiliary input terminals that are connected to an impedance measuring circuit 50. The switch array has the ability to connect the two output terminals of the impedance measuring circuit 50 to either (a) the first and second output connectors, (b) the first and third output connectors, (c) the first and fourth output connectors, (d) the second and third output connectors, (e) the second and fourth output connectors, or (f) the third and fourth output connectors. The switch array 40 implements one of the options (a)-(f) described in the previous sentence based on at least one control signal that arrives from the controller 30.

The switch array 40 can be implemented using an array of electromechanical relays, an array of solid state switches, etc. And the internal configuration of the switch array that enables it to perform the functions described in the previous paragraphs will be apparent to persons skilled in the relevant arts.

The controller 30 can be a microcontroller or a microprocessor that has been programmed to implement the functions described herein, and the program that runs on the controller 30 can be stored in in computer-readable media. The computer-readable media can be e.g., a non-volatile memory such as ROM, PROM, EPROM, solid state drives, hard drives, etc. Additionally or alternatively, the controller 30 can be implemented in hardware including but not limited to one or more application-specific integrated circuits (ASICs).

The impedance measuring circuit 50 sends auxiliary signals into whichever output connectors it is hooked up to (which will depend on the current state of the switch array 40), and the impedance measuring circuit 50 measures the impedance that is encountered by those auxiliary signals. For example, if the switch array 40 has connected the two output terminals of the impedance measuring circuit 50 to the first and second output connectors, the impedance measuring circuit 50 will measure the impedance between the first and second output connectors. And because (in the example depicted in FIG. 2) the first and second output connectors lead to the posterior and right electrode assemblies 10, respectively, the impedance measuring circuit 50 will measure the impedance that the subject's body presents between the posterior and right electrode assemblies 10.

Similarly, if the switch array 40 has connected the two output terminals of the impedance measuring circuit 50 to the first and third output connectors, the impedance measuring circuit 50 will measure the impedance between the first and third output connectors. And because (in the example depicted in FIG. 2) the first and third output connectors lead to the posterior and anterior electrode assemblies 10, respectively, the impedance measuring circuit 50 will measure the impedance that the subject's body presents between the posterior and anterior electrode assemblies.

Because four electrode assemblies 10 are connected to the first, second, third, and fourth output connectors of the switch array 40 via first, second, third, and fourth cables, respectively, the switch array 40 can set up the connections so that the impedance measuring circuit 50 can measure the impedance between all possible combinations of the electrode assemblies 10. This means that, regardless of which of the four electrode assemblies 10 are connected to which of the four connectors of the switch array 40, the internal connections within the switch array 40 can be set (based on at least one control signal that arrives from the controller 30) so that the impedance measuring circuit 50 can measure the impedances (a) between the anterior and posterior electrode assemblies, (b) between the anterior and left electrode assemblies, (c) between the anterior and right electrode assemblies, (d) between the left and right electrode assemblies, (e) between the left and posterior electrode assemblies, and (f) between the right and posterior electrode assemblies.

The impedance measuring circuit 50 can measure the impedance between whichever electrode assemblies 10 it is hooked up to using a variety of approaches that will be apparent to persons skilled in the relevant arts, including but not limited to (a) sending a known current (e.g., at 200 kHz) into whichever electrode assemblies 10 it is hooked up to and measuring the resulting voltage or (b) sending a known voltage (e.g., at 200 kHz) into whichever electrode assemblies 10 it is hooked up to and measuring the resulting current. In either case, the impedance measuring circuit 50 will be sending auxiliary signals into two of the electrode assemblies 10 (via corresponding output connectors of the switch array 40), and accepting input data (via the same connectors) indicative of the effects of sending those signals into those two electrode assemblies 10.

As explained above, the switch array 40 has the ability to connect the two output terminals of the impedance measuring circuit 50 to either (a) the first and second output connectors, (b) the first and third output connectors, (c) the first and fourth output connectors, (d) the second and third output connectors, (e) the second and fourth output connectors, or (f) the third and fourth output connectors.

So in some embodiments, the controller 30 is programmed to sequentially set the switch array to each of these states in turn, and command the impedance measuring circuit 50 to measure the impedance during each of these states. This will return six impedance measurements. Based on these six measurements, the controller 30 can determine which of the electrode assemblies are positioned on opposite sides of the region of interest in the subject's body. This works because the impedance between electrode assemblies that are positioned on opposite sides of a subject's body (e.g., between the left and right electrode assemblies 10 or between the anterior and posterior electrode assemblies 10) will be higher than the impedance between electrode assemblies that are adjacent to each other (e.g., between the left and anterior electrode assemblies 10 or between the right and posterior electrode assemblies 10) due to the lengths of the corresponding paths through the subject's body that must be traversed by the electrical signals.

After the controller 30 has determined which of the electrode assemblies are positioned on opposite sides of the region of interest, the controller sends at least one control signal to the switch array 40 which causes the switch array to route the output of the AC signal generator 20 to connectors that lead, via corresponding cables, to electrode assemblies 10 that are positioned on opposite sides of the region of interest. The controller 30 can implement this, for example, by (i) controlling the switch array 40 so that the switch array routes the output of the AC signal generator 20 to connectors that lead, via corresponding cables, to a first pair of the electrode assemblies 10 that are positioned on opposite sides of the region of interest, (ii) controlling the switch array 40 so that the switch array routes the output of the AC signal generator 20 to connectors that lead, via corresponding cables, to a second pair of the electrode assemblies that are positioned on opposite sides of the region of interest, and repeating steps (i) and (ii) in an alternating sequence.

Two concrete examples may be helpful to clarify this sequence of events. If the electrode assemblies 10 that are positioned on a person's head are hooked up to the switch array 40 in the configuration depicted in FIG. 2 (i.e., with the posterior, right, anterior, and left electrode assemblies connected to connectors #1, #2, #3, and #4, respectively), and all six possible impedance measurements are made as described above, the two highest impedance measurements will be (i) the impedance between connectors #1 and #3 and (ii) the impedance between connectors #2 and #4. (This is due to the longer lengths of the corresponding paths through the subject's body that must be traversed by the electrical signals that travel via those connectors.) Based on these impedance measurements, the controller can determine that connectors #1 and #3 lead to one pair of electrode assemblies 10 that are positioned on opposite sides of the region of interest, and that connectors #2 and #4 lead to a second pair of electrode assemblies 10 that are positioned on opposite sides of the region of interest.

The controller 30 then sends at least one control signal to the switch array 40 which causes the switch array to route the output of the AC signal generator 20 to connectors that lead, via corresponding cables, to electrode assemblies 10 that are positioned on opposite sides of the region of interest. The controller 30 can implement this, for example, by (i) controlling the switch array 40 so that the switch array routes the output of the AC signal generator 20 to connectors #1 and #3 (which lead, via corresponding cables, to the posterior and anterior electrode assemblies 10), (ii) controlling the switch array 40 so that the switch array routes the output of the AC signal generator 20 to connectors #2 and #4 (which lead, via corresponding cables, to the right and left electrode assemblies), and repeating steps (i) and (ii) in an alternating sequence.

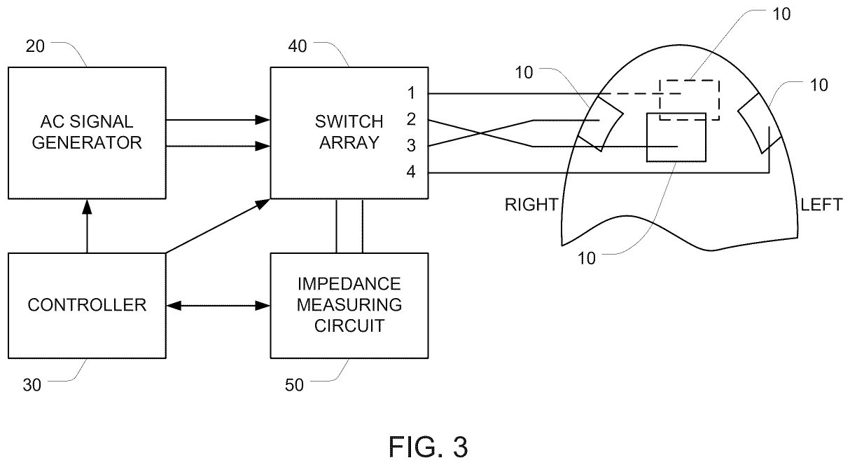

The second concrete example is identical to the first concrete example, except that the position of the connectors from the electrode assemblies 10 that were plugged into connectors #2 and #3 of the switch array 40 are swapped. Thus, in the second concrete example, the electrode assemblies 10 on the person's head are hooked up to the switch array 40 in the configuration depicted in FIG. 3 (i.e., with the posterior, anterior, right, and left electrode assemblies connected to connectors #1, #2, #3, and #4, respectively). When all six possible impedance measurements are made as described above, the two highest impedance measurements will be (i) the impedance between connectors #1 and #2 and (ii) the impedance between connectors #3 and #4 (due to the longer path lengths through the subject's body). Based on these impedance measurements, the controller can determine that connectors #1 and #2 lead to one pair of electrode assemblies 10 that are positioned on opposite sides of the region of interest, and that connectors #3 and #4 lead to a second pair of electrode assemblies 10 that are positioned on opposite sides of the region of interest.

The controller 30 then sends at least one control signal to the switch array 40 which causes the switch array to route the output of the AC signal generator 20 to connectors that lead, via corresponding cables, to electrode assemblies 10 that are positioned on opposite sides of the region of interest. The controller 30 can implement this, for example, by (i) controlling the switch array 40 so that the switch array routes the output of the AC signal generator 20 to connectors #1 and #2 (which lead, via corresponding cables, to the posterior and anterior electrode assemblies 10), (ii) controlling the switch array 40 so that the switch array routes the output of the AC signal generator 20 to connectors #3 and #4 (which lead, via corresponding cables, to the right and left electrode assemblies), and repeating steps (i) and (ii) in an alternating sequence.

These two concrete examples illustrate how, regardless of which electrode assembly is positioned on which part of the subject's body, the controller 30 can determine which electrode assemblies 10 are positioned opposite to each other, and dynamically configure the application of the alternating voltages so that alternating voltages are applied between electrode assemblies that are disposed on opposite sides of the region of interest.

While the examples discussed above in connection with FIGS. 2-3 describe relying on six impedance measurements to determine which of the electrode assemblies are positioned on opposite sides of a region of interest in the subject's body, it will often be possible to make that determination based on fewer than six impedance measurements (e.g., five, four, or possibly even three impedance measurements in certain circumstances). In addition, as soon as the controller 30 determines that one pair of electrode assemblies 10 are positioned on opposite sides of a region of interest, the controller 30 can conclude (by process of elimination) that the remaining pair of electrode assemblies 10 are also positioned on opposite sides of the region of interest.

In a variation of the embodiments described above in connection with FIG. 2-3, a conductance measuring circuit can be used in place of the illustrated impedance measuring circuit 50, in which case the controller 30 relies on the lowest conductance readings instead of the highest impedance readings.

FIG. 4 is a block diagram of a second embodiment that relies on sending signals into the electrode assemblies 10′ that are positioned on the subject's body. These signals cause light sources 15 that are incorporated within those electrode assemblies 10′ to illuminate. Observations of the effects of these signals (i.e., which light sources turn on) is used to determine which of the electrode assemblies are positioned on opposite sides of a region of interest in the subject's body. This determination is used to dynamically configure how alternating voltages are applied to the set of electrode assemblies 10′.

The electrode assemblies 10′ in this embodiment are similar to the electrode assemblies in the FIG. 2 embodiment, except that each electrode assembly 10′ includes a light source 15 that turns on in response to a signal that arrives at the electrode assembly 10′ via a dedicated wire within the cable that leads to the electrode assembly and plugs into the switch array 140.

The AC signal generator 20 in this FIG. 4 embodiment is similar to the AC signal generator 20 described above in connection with FIG. 2.

A switch array 140 has two primary input terminals and four output connectors, and it routes the signals that arrive (from the AC generator 20) at the two primary input terminals to either (a) the first and second output connectors, (b) the first and third output connectors, (c) the first and fourth output connectors, (d) the second and third output connectors, (e) the second and fourth output connectors, or (f) the third and fourth output connectors. The switch array 140 implements one of the options (a)-(f) described in the previous sentence based on at least one control signal that arrives from a controller 30.

The switch array 140 also has an auxiliary input terminal that is connected to an LED driver circuit 150. The switch array has the ability to route the output of the LED driver circuit 150 to either (a) a pin within the first connector dedicated to driving an LED, (b) a pin within the second output connector dedicated to driving an LED, (c) a pin within the third output connector dedicated to driving an LED, or (d) a pin within the fourth output connector dedicated to driving an LED. The switch array 140 implements one of the options (a)-(d) described in the previous sentence based on at least one control signal that arrives from the controller 30.

The switch array 140 can be implemented using an array of electromechanical relays, an array of solid state switches, etc. And the internal configuration of the switch array that enables it to perform the functions described in the previous paragraphs will be apparent to persons skilled in the relevant arts.

The controller 30 can be a microcontroller or a microprocessor or can be implemented in hardware including but not limited to one or more application-specific integrated circuits (ASICs), as described above in connection with FIG. 2.

The controller 30 commands the LED driver circuit 150 to send an auxiliary signal into whichever output connector it is hooked up to (which will depend on the current state of the switch array 140). When this signal is applied to any of the connectors, the output of the LED driver circuit 150 will traverse the corresponding connector pin, travel through the cable, and cause the LED 15 on the corresponding one of the electrode assemblies 10′ to light up.

Because four electrode assemblies 10′ are connected to the first, second, third, and fourth output connectors of the switch array 140 via first, second, third, and fourth cables, respectively, the switch array 140 and the LED driver circuit 150 can collectively cause any of the LEDs 15 located on the electrode assemblies 10′ to light up.

While the LEDs 15 on the electrode assemblies 10′ are being activated in this manner, a camera 160 captures images of the electrode assemblies 10′ and sends those images to the controller 30. The controller 30 analyzes those images, and by observing which LED is lit up in response to sending the LED driving signal into any of the outputs of the switch array 140, the controller 30 can determine which of the four electrode assemblies 10′ is connected to which of the outputs of the switch array 140.

This will now be explained by the following examples. Assume, in a first example, that four electrode assemblies 10′ are positioned on a person's head and are hooked up to the switch array 140 in the configuration depicted in FIG. 4 (i.e., with the posterior, right, anterior, and left electrode assemblies connected to connectors #1, #2, #3, and #4, respectively). Under these circumstances, (a) if the switch array 140 is set to route the output of the LED driver circuit 150 to the switch array's first output, it will cause the LED 15 on the posterior electrode assembly 10′ to light up; (b) if the switch array 140 is set to route the output of the LED driver circuit 150 to the switch array's second output, it will cause the LED 15 on the right electrode assembly 10′ to light up; (c) if the switch array 140 is set to route the output of the LED driver circuit 150 to the switch array's third output, it will cause the LED 15 on the anterior electrode assembly 10′ to light up; and (d) if the switch array 140 is set to route the output of the LED driver circuit 150 to the switch array's fourth output, it will cause the LED 15 on the left electrode assembly 10′ to light up.

The controller 30 analyzes the images of the electrode assemblies 10′ while the LED driver circuit 150 is activated during each of the four conditions (a)-(d) described in the previous paragraph. Based on these images (combined with suitable image recognition software the nature of which will be apparent to persons skilled in the relevant arts), the controller 30 can determine which electrode assembly 10′ is connected to which output of the switch array 140. For example, the image recognition software could identify whether ears and eyes that appear in the image, and conclude that (a) the LEDs that are closest to the two identified ears must reside on electrode assemblies 10′ that are positioned on the right and left sides of the subject's head; (b) the LED that is positioned closest to the two identified eyes must reside on an electrode assembly 10′ that is positioned in front of the subject's head; and (c) the LED that is positioned furthest from the identified eyes and ears must reside on an electrode assembly 10′ that is positioned in the back of the subject's head.

Note that it is not necessary for the camera 160 to visualize all four of the electrode assemblies 10′ to determine which electrode assemblies are positioned on opposite sides of the region of interest. To the contrary-the camera 160 only needs to visualize three of the electrode assemblies to make that determination. For if the camera can visualize three electrode assemblies, at least two of those electrode assemblies will be positioned on opposite sides of the region of interest. The controller 30 can then conclude (by a process of elimination) that the remaining two electrode assemblies are also positioned on opposite sides of the region of interest.

Moreover, the three electrode assemblies that are visualized by the camera need not all appear in a single image frame that is captured by the camera, if the camera 160 captures multiple images of the electrode assemblies 10′ while the subject moves their body. Assume, for example, that the camera 160 depicted in FIG. 4 can only visualize the right and anterior electrode assemblies 10′. By controlling the switch array 140, activating the LED driver circuit 150, and observing the images captured by the camera 160, the controller 30 can ascertain that those two electrode assemblies 10′ are adjacent to each other and therefore are not positioned on opposite sides of the region of interest. When the subject turns their head to the right, the camera 160 will be able to visualize the anterior and left electrode assemblies. By controlling the switch array 140, activating the LED driver circuit 150, and observing the images captured by the camera 160, the controller 30 can ascertain that those two electrode assemblies 10′ are also adjacent to each other and therefore are not positioned on opposite sides of the region of interest. Controller 30 will then be able to determine, by process of elimination, that the anterior electrode assembly and the electrode assembly that has not yet been visualized (i.e., the posterior electrode assembly) are positioned on opposite sides of the region of interest. And the controller 30 can then subsequently determine by process of elimination that the remaining two electrode assemblies 10′ (i.e., the right and left electrode assemblies) are also positioned on opposite sides of the region of interest.

After the controller 30 has determined which of the electrode assemblies are positioned on opposite sides of the region of interest, the controller sends at least one control signal to the switch array 140 which causes the switch array to route the output of the AC signal generator 20 to connectors that lead, via corresponding cables, to electrode assemblies 10′ that are positioned on opposite sides of the region of interest. The controller 30 can implement this, for example, by (i) controlling the switch array 140 so that the switch array routes the output of the AC signal generator 20 to connectors that lead, via corresponding cables, to a first pair of the electrode assemblies 10′ that are positioned on opposite sides of the region of interest, (ii) controlling the switch array 140 so that the switch array routes the output of the AC signal generator 20 to connectors that lead, via corresponding cables, to a second pair of the electrode assemblies that are positioned on opposite sides of the region of interest, and repeating steps (i) and (ii) in an alternating sequence.

If the cables between the electrode assemblies 10′ and the switch array 140 are hooked up as depicted in FIG. 4, the controller 30 can implement this by (i) controlling the switch array 140 so that the switch array routes the output of the AC signal generator 20 to connectors #1 and #3 (which lead, via corresponding cables, to the posterior and anterior electrode assemblies 10′), (ii) controlling the switch array 140 so that the switch array routes the output of the AC signal generator 20 to connectors #2 and #4 (which lead, via corresponding cables, to the right and left electrode assemblies 10′), and repeating steps (i) and (ii) in an alternating sequence.

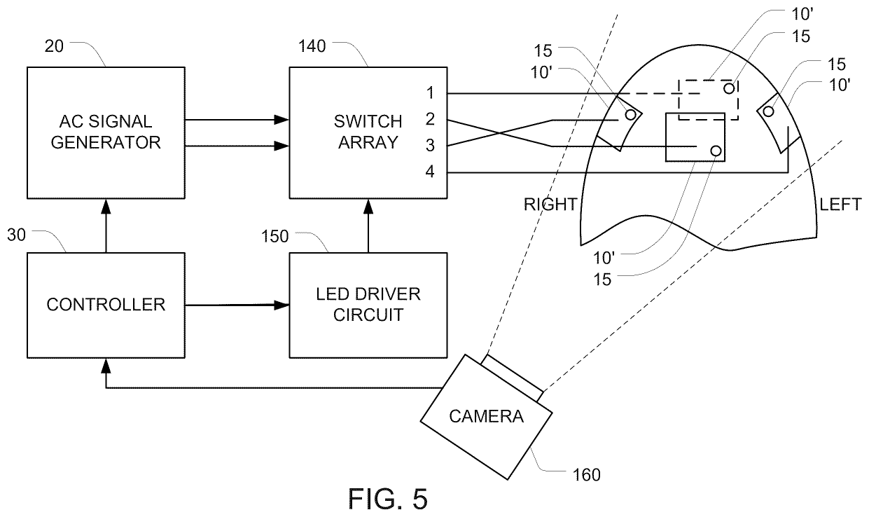

Alternatively, if the cables between the electrode assemblies 10′ and the switch array 140 are hooked up as depicted in FIG. 5, The controller 30 can implement this by (i) controlling the switch array 140 so that the switch array routes the output of the AC signal generator 20 to connectors #1 and #2 (which lead, via corresponding cables, to the posterior and anterior electrode assemblies 10′), (ii) controlling the switch array 140 so that the switch array routes the output of the AC signal generator 20 to connectors #3 and #4 (which lead, via corresponding cables, to the right and left electrode assemblies 10′), and repeating steps (i) and (ii) in an alternating sequence.

These two examples illustrate how, regardless of which electrode assembly 10′ is positioned on which part of the subject's body, the controller 30 can determine which electrode assemblies 10′ are positioned opposite to each other, and dynamically configure the application of the alternating voltages so that alternating voltages are applied between electrode assemblies that are disposed on opposite sides of the region of interest.

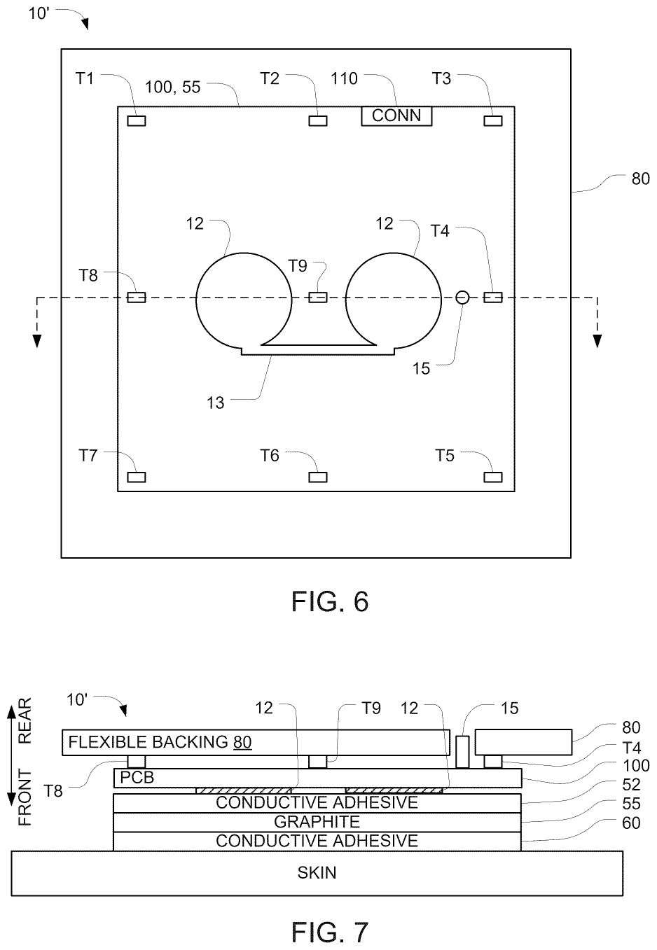

FIGS. 6 and 7 depict plan and section views of one example of how to implement the electrode assemblies 10′ described above in connection with FIGS. 4-5. Each electrode assembly 10′ includes a flexible PCB 100 (i.e., a flex circuit) that has at least one metal pad 12 (e.g., copper) disposed on its front face, and a plurality of conductive traces (not shown) that are also disposed on the flex circuit 100.

At least one intermediate layer is disposed in front of the flex circuit 100. In the embodiment depicted in FIGS. 6 and 7, the at least one intermediate layer includes a first layer of conductive adhesive 52 and a layer of graphite 55. In alternative embodiments (not shown), the at least one intermediate layer could comprise a layer of flexible polymer material with a dielectric constant of at least 10.

The first layer of conductive adhesive 52 is disposed on and in front of the flex circuit 100. The rear surface of the first layer of conductive adhesive 52 will adhere to the front surface of the metal pads 12 and may also contact some portion(s) of the flex circuit 100.

A layer of graphite 55 is disposed on and in front of the first layer of conductive adhesive 52, and a second layer of conductive adhesive 60 is disposed on and in front of the layer of graphite 55. The layer of graphite 55 can be, e.g., a layer of pyrolytic graphite, graphitized polymer film, or graphite foil made from compressed high purity exfoliated mineral graphite. The layer of graphite 55 acts to spread both the flow of current and heat in all four directions (i.e., to the right, to the left, into the page, and out of the page in FIG. 7, which corresponds to right, left, up, and down in FIG. 6).

A second layer of conductive adhesive 60 is disposed on the front face of the layer of graphite. This layer of conductive adhesive 60 should be biocompatible, and its function is to hold the electrode assembly 10′ against the subject's skin. Note that in alternative embodiments, a layer of conductive gel (e.g., hydrogel) can be used in place of the second layer of conductive adhesive 60 depicted in FIG. 7.

A plurality of temperature sensors (e.g., thermistors) T1-T9 are mounted to the flex circuit, and these temperature sensors are disposed in thermal contact (either direct thermal contact or indirect thermal contact) with the at least one metal pad. In those embodiments that include a layer of graphite, the temperature sensors can be disposed in thermal contact with a layer of graphite instead of being disposed in thermal contact with the at least one metal pad.

A flexible backing 80 (e.g., a bandage-like backing) is positioned behind the PCB 100, and this flexible backing 80 is configured to support the PCB. In the embodiment depicted in FIGS. 6-7, a portion of the flexible backing 80 extends laterally beyond the flex circuit and the front of this portion may be covered with a biocompatible adhesive that adheres to skin. This portion of the flexible backing 80 helps hold the electrode assembly 10′ against the subject's skin. In other embodiments (not shown), the flexible backing does not extend laterally beyond the flex circuit 100, and the electrode assembly 10′ may be supported in position on the subject's skin solely by the conductive adhesive 60.

An LED 15 is mounted to the flex circuit 100, and it is aimed in a rear direction through a hole in the flexible backing 80 so that when the LED 15 is activated, it will be visible while the electrode assembly 10′ is positioned on a subject's body.

A connector 110 is mounted to the flex circuit 100, and this connector 110 is used to provide an electrical interface with the at least one metal pad 12, the thermistors T1-T9 and the LED 15. One end of the cable that leads to the electrode assembly 10′ (described above in connection with FIGS. 4-5) terminates on this connector 110.

The other end of the cable has a multi-pin connector that mates with any of the outputs of the switch array 140 described above in connection with FIGS. 4-5. The cable is flexible and has a first conductor configured to route first electrical signals from a first pin of the multi-pin connector to the at least one conductive pad 12. These first electrical signals are configured to, when at least two electrode assemblies 10′ have been affixed to a subject's body, induce an alternating electric field in a region of interest in the subject's body. The cable also has at least one second conductor configured to route second electrical signals from the at least one temperature sensor T1-T9 to at least one second pin of the multi-pin connector. And the cable also has at least one third conductor configured to route third electrical signals from at least one third pin of the multi-pin connector to the LED 15. The third electrical signals are configured to cause the LED 15 to emit light. Note that as used herein, the term “pin” could be either a male pin or a female pin.

Note that the electrode assembly described above in connection with FIGS. 6-7 is not the only variety of electrode assembly into which an LED can be incorporated. To the contrary, an LED can be added to any electrode assembly that is suitable for applying TTFields to a subject's body, as long as the LED is positioned and oriented so that the light that it emits can be seen by observers in the vicinity of the subject.

Thus, the example depicted in FIGS. 6 and 7 can be generalized to the following: An electrode assembly that comprises at least one electrode element positioned and oriented to apply alternating electric fields to a subject's body; a layer of conductive adhesive or conductive hydrogel that is (i) positioned in front of the at least one electrode element and (ii) configured to adhere to a subject's body; at least one temperature sensor disposed in direct and/or indirect thermal contact with the at least one electrode element; a light source; and a flexible cable. The light source is positioned and oriented so that light emitted by the light source can be seen by observers in a vicinity of the subject while the electrode assembly is positioned on the subject's body. The flexible cable has (a) a multi-pin connector, (b) a first conductor configured to route first electrical signals from a first pin of the connector to the at least one electrode element, wherein the first electrical signals are configured to, when at least two electrode assemblies have been affixed to a subject's body, induce an alternating electric field in a region of interest in the subject's body, (c) at least one second conductor configured to route second electrical signals from the at least one temperature sensor to at least one second pin of the connector, and (d) at least one third conductor configured to route third electrical signals from at least one third pin of the connector to the light source, wherein the third electrical signals are configured to cause the light source to emit light. Optionally, at least one intermediate layer of material (including but not limited to a layer of graphite, a layer of ceramic material with a dielectric constant of at least 1000, or a layer of a flexible polymer with a dielectric constant of at least 10) can be positioned between the at least one electrode element and the layer of conductive adhesive or conductive hydrogel.

Note also that while FIGS. 2-5 each depicts four electrode assemblies 10/10′, a different number of electrode assemblies (e.g., 6) may be positioned on the subject's body, and the concepts described above in connection with FIGS. 2-5 may be used to determine which electrode assemblies are disposed on opposite sides of the ROI. Furthermore, a determination of which electrode assemblies are disposed on opposite sides of the ROI is not limited to determining which *single* electrode assembly is opposite to another *single* electrode assembly. Assume, for example, that six electrode assemblies are positioned around a region of interest at radial positions of 0°, 60°, 120°, 180°, 240°, 300°, and 360°. Using the concepts described above, the controller 30 can determine that the electrode assembly positioned at 0° is opposite to the *set* of three electrode assemblies located at the 120°, the 180°, and the 240° positions. The controller 30 can then command the switch array 40/140 to route one phase of the AC signal generator's 20 output to the electrode assembly at the 0° position, and to route the other phase of the AC signal generator's 20 output to the entire set of three electrode assemblies (i.e., the electrode assemblies positioned at 120°, 180°, and 240°).

Finally, it is important to note that the usage of the identifiers (a), (b), (c), (d), etc. in the claims below does not imply a particular sequence in time for the corresponding steps. For while it is certainly possible that step (a) will precede step (b) in time, different sequencings of those steps are also possible, except in cases where a particular sequencing is inconsistent with the internal language of the various steps or with other language in the claims. For example, a step labeled (b) could precede a step labeled (a) in time. It is also possible for two or more steps to occur simultaneously or to overlap to an extent, except in cases where simultaneity or overlapping would be inconsistent with the internal language of the various steps or with other language in the claims.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method of configuring a system that includes at least four electrode assemblies positioned on or in a subject's body, wherein each of the electrode assemblies is connected via a respective cable to a switch array, the method comprising:
sending at least one first signal into a first subset of the cables;
observing at least one first effect of the sending of the at least one first signal;
sending at least one second signal into a second subset of the cables;
observing at least one second effect of the sending of the at least one second signal;
sending at least one third signal into a third subset of the cables;
observing at least one third effect of the sending of the at least one third signal; and
determining, based on the observed first, second, and third effects, which of the electrode assemblies are positioned on opposite sides of a region of interest in the subject's body.

2. The method of claim 1, further comprising configuring the switch array to route an AC voltage between whichever electrode assemblies were determined to be positioned on opposite sides of the region of interest.

3. The method of claim 2, further comprising applying the AC voltage between whichever electrode assemblies were determined to be positioned on opposite sides of the region of interest.

4. The method of claim 1, further comprising:
(a) applying an AC voltage between a first pair of the electrode assemblies that was determined to be positioned on opposite sides of the region of interest;
(b) applying an AC voltage between a second pair of the electrode assemblies that was determined to be positioned on opposite sides of the region of interest; and
(c) repeating step (a) and step (b) in an alternating sequence at least 100 times.

5. The method of claim 1, further comprising:
sending at least one fourth signal into a fourth subset of the cables; and
observing at least one fourth effect of the sending of the at least one fourth signal,
wherein the determining is based on the observed first, second, third, and fourth effects.

6. The method of claim 1, wherein the determining is based on detecting light that is emitted in response to the sending of the at least one first signal, the at least one second signal, and the at least one third signal.

7. The method of claim 1, wherein each of the electrode assemblies includes a respective light source,
wherein the sending of the at least one first signal into the first subset of the cables comprises activating a first single one of the light sources via a first single one of the cables, and wherein the observing of the at least one first effect comprises observing which of the light sources is emitting light,
wherein the sending of the at least one second signal into the second subset of the cables comprises activating a second single one of the light sources via a second single one of the cables, and wherein the observing of the at least one second effect comprises observing which of the light sources is emitting light,
wherein the sending of the at least one third signal into the third subset of the cables comprises activating a third single one of the light sources via a third single one of the cables, and wherein the observing of the at least one third effect comprises observing which of the light sources is emitting light.

8. The method of claim 7, wherein the observing is implemented by capturing a plurality of images of at least some of the electrode assemblies using at least one camera, and
wherein the determining is implemented by analyzing the captured plurality of images.

9. The method of claim 1, wherein the determining is based on measuring impedances or conductances that are encountered by the at least one first signal, the at least one second signal, and the at least one third signal.

10. The method of claim 1, wherein the sending of the at least one first signal into the first subset of the cables comprises applying a first electrical signal between a first single one of the cables and a second single one of the cables, and wherein the observing of at least one first effect of the sending of the at least one first signal comprises measuring an impedance that is encountered by the first electrical signal or measuring a conductance that is encountered by the first electrical signal,
wherein the sending of the at least one second signal into the second subset of the cables comprises applying a second electrical signal between the first single one of the cables and a third single one of the cables, and wherein the observing of at least one second effect of the sending of the at least one second signal comprises measuring an impedance that is encountered by the second electrical signal or measuring a conductance that is encountered by the second electrical signal,
wherein the sending of the at least one third signal into the third subset of the cables comprises applying a third electrical signal between the first single one of the cables and a fourth single one of the cables, and wherein the observing of at least one third effect of the sending of the at least one third signal comprises measuring an impedance that is encountered by the third electrical signal or measuring a conductance that is encountered by the third electrical signal.

11. An apparatus for applying alternating electric fields to a region of interest within a subject's body, the apparatus comprising:
a controller;
an AC signal generator that generates an output at a frequency between 50 kHz and 1 MHz;
a switch array having (i) two inputs that accept the output of the AC signal generator and (ii) first, second, third, and fourth output connectors; and
a circuit configured to send auxiliary signals into the first, second, third, and fourth output connectors when electrode assemblies are connected to the first, second, third, and fourth output connectors via first, second, third, and fourth cables, respectively,
wherein the switch array is dynamically configurable to, based on at least one control signal that arrives from the controller, route the output of the AC signal generator to either (a) the first and second output connectors, (b) the first and third output connectors, (c) the first and fourth output connectors, (d) the second and third output connectors, (e) the second and fourth output connectors, or (f) the third and fourth output connectors; and
wherein the controller is configured to (a) accept input data indicative of effects of the sending of the auxiliary signals into the first, second, third, and fourth output connectors, (b) determine, based on the input data, which of the electrode assemblies are positioned on opposite sides of a region of interest in the subject's body, and (c) control the switch array so that the switch array routes the output of the AC signal generator to connectors that lead, via corresponding cables, to electrode assemblies that are positioned on opposite sides of the region of interest.

12. The apparatus of claim 11, wherein the controller is configured to
(i) control the switch array so that the switch array routes the output of the AC signal generator to connectors that lead, via corresponding cables, to a first pair of the electrode assemblies that are positioned on opposite sides of the region of interest,
(ii) control the switch array so that the switch array routes the output of the AC signal generator to connectors that lead, via corresponding cables, to a second pair of the electrode assemblies that are positioned on opposite sides of the region of interest, and
repeat steps (i) and (ii) in an alternating sequence.

13. The apparatus of claim 11, wherein the auxiliary signals that are sent into the first, second, third, and fourth output connectors are configured to activate respective light sources that are positioned on the electrode assemblies.

14. The apparatus of claim 13, wherein the accepted input data represent at least one image of the electrode assemblies.

15. The apparatus of claim 11, wherein the auxiliary signals that are sent into the first, second, third, and fourth output connectors are configured to obtain impedance measurements or conductance measurements, and wherein the accepted input data represent impedance measurements or conductance measurements.

16. An electrode assembly comprising:
at least one electrode element positioned and oriented to apply alternating electric fields to a subject's body;
a layer of conductive adhesive or conductive hydrogel that is (i) positioned in front of the at least one electrode element and (ii) configured to adhere to a subject's body;
at least one temperature sensor disposed in direct and/or indirect thermal contact with the at least one electrode element;
a light source positioned and oriented so that light emitted by the light source can be seen by observers in a vicinity of the subject while the electrode assembly is positioned on the subject's body; and
a flexible cable having a (a) a multi-pin connector, (b) a first conductor configured to route first electrical signals from a first pin of the connector to the at least one electrode element, wherein the first electrical signals are configured to, when at least two electrode assemblies have been affixed to a subject's body, induce an alternating electric field in a region of interest in the subject's body, (c) at least one second conductor configured to route second electrical signals from the at least one temperature sensor to at least one second pin of the connector, and (d) at least one third conductor configured to route third electrical signals from at least one third pin of the connector to the light source, wherein the third electrical signals are configured to cause the light source to emit light.

17. The electrode assembly of claim 16, further comprising at least one intermediate layer of material positioned between the at least one electrode element and the layer of conductive adhesive or conductive hydrogel.

18. The electrode assembly of claim 17, wherein the at least one intermediate layer comprises:
a sheet of graphite disposed in front of the at least one electrode element, wherein the sheet of graphite has a front face and a rear face; and
a layer of conductive adhesive disposed between the at least one electrode element and the rear face of the sheet of graphite, in electrical contact with both the at least one electrode element and the rear face of the sheet of graphite.

19. The electrode assembly of claim 17, wherein the at least one intermediate layer comprises a layer of flexible polymer material having a dielectric constant of at least 10.

20. The electrode assembly of claim 16, wherein the at least one electrode element comprises a conductive pad that is disposed on a front side of a flex circuit.
